(19) United States
(12) Patent Application Publication
(10) Pub. No.: US 2005/0277826 A1
(43) Pub. Date: Dec. 15, 2005

(54) Apparatus and method for reducing interference

(75) Inventor: William James Ross Dunseath JR., Silver City, NM (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc.

(21) Appl. No.: 10/865,446

(22) Filed: Jun. 10, 2004

(51) Int. Cl. A61B 5/05

(52) U.S. Cl. 600/410

(57) ABSTRACT

In an electronic apparatus and method for reducing interference in an EPM signal, there is provided (a) a signal line connected to a-signal electrode; and (b) a reference line connected to a reference electrode. The signal line and reference line are associated by being in close physical proximity for a substantial part of their lengths. Subtraction means subtracts an interference signal on the reference line from an interference signal on the signal line to enhance a desired signal on the signal line.

### FIELD OF THE INVENTION

This present invention relates to an electronic method and apparatus for reducing interference in a signal wherein the interference is of a large magnitude relative to the data component to be extracted from the signal. It is particularly, although not exclusively, suited to reducing noise in biopotential signal acquisition, which noise is caused by electrical and magnetic fields.

### BACKGROUND OF THE INVENTION

Functional magnetic resonance imaging (fMRI) is widely used in both medical and non-medical imaging to obtain a spatial image of “slices” through the brain. In the medical context, it can be used to identify lesions such as areas of restricted blood flow or tumours. Outside the medical field, it has, for example, been a useful tool in cognitive neuroscience for investigating brain response to various external stimuli.

Electroencephalography (EEG) has traditionally been used for investigations into brain activity. It may, for example, be used to investigate abnormal brain activity in disease states such as epilepsy or in certain psychiatric abnormalities.

If fMRI and EEG could be used together, they could advantageously combine both spatial and temporal information which would be of major benefit for both medical and non-medical uses. However, a typical EEG signal obtained from a scalp electrode is in the range typically of 1 μV to 100 μV at an impedance of around 500 Ω to 50K Ω. The large magnetic and radio frequency (rf) fields produced by MRI machines easily swamp this signal with induced noise on the signal wire.

Conventional known methods for rejecting interference in EEG include the use of a reference electrode and differential amplifier, electrical isolation of the EEG amplifiers, shielding of the electrode lead wires, driving the shield of the lead wires with a common mode voltage, and electrical filtering of the EEG signal. Additional strategies have been employed for EEG in fMRI, such as the use of carbon lead wires and inductors.

For example, U.S. Pat. No. 5,445,162 proposes a system using electrodes and wiring designed to minimise noise pick-up and the fMRI and EEG data are obtained alternately. Thus, although the system purports to enable fMRI and EEG signals to be obtained at the same time from an individual, the technique does not permit obtaining truly simultaneous fMRI and EEG data. However, it does propose locating the EEG recording equipment outside the MRI room to minimise interference. In fact, to date, there has been no disclosure of a method for obtaining a clean EEG during the actual scanning performed in fMRI.

Various claims have been made for “simultaneous” EEG in fMRI, but this has meant only that EEG's have been obtained while the subject is in the fMRI apparatus, not when it is actually scanning. An object of the invention is to obtain true simultaneous EEG (or other electrophysiological measurement) and fMRI without resorting to methods that distort or change the standard multi-channel EEG electrode configurations and relationships.

WO-A-03/073929 discusses the potential problems associated with concurrent fMRI and EEG measurements, namely noise induced in the EEG signal by the rf and magnetic fields (as mentioned above) and the disruption to the fMRI measurement by introduction of ferromagnetic material in the EEG electrodes, into the bore of the fMRI machine. This reference comments upon possibilities for alleviating these problems. One is to dispense with ferromagnetic materials in the EEG electrodes and to use an alternative such as carbon fibre. Another is to rearrange the EEG leads to minimise interference with the rf field.

The aforementioned WO-A-03/073929 also recognises safety problems inherent in deploying EEG equipment inside a pulsed rf field, eg due to induced currents. Solutions to these problems have included raising the impedance of the EEG detection circuit by means of resistors or by using different electrode systems on different electrode materials, or by incorporating a fibre optic link in the line between the electrodes and the circuit. The reference proposes that a better method of avoiding such hazards is to incorporate an amplifier within the electrode structure.

Despite these numerous proposals, there still remains a need for a system whereby truly simultaneous derivation of EEG and fMRI signals could be made possible, especially having the ability to conduct the totality of measurement within the MRI room.

In principle, any one of a number of electrophysiological measurement systems can be combined with fMRI, instead of or in addition to EEG. These are electrocardiography (ECG), electromyography (EMG), electro-oculography (EOG), electroretinography (ERG) and galvanic skin response measurement (GSR). The same problems can occur with any electrophysiological measurement such as these, when used in combination with fMRI. Therefore, the problem to be solved may be stated as the need to suppress interference sufficiently when simultaneously conducting any electrophysiological measurement in combination with fMRI. For convenience, for the generic term electrophysiological measurement, hereinafter the abbreviation EPM will be used.

We have now devised an electronic noise reduction system which solves this problem.

### DEFINITION OF THE INVENTION

A first aspect of the present invention now provides an electronic apparatus for reducing interference in a desired signal, the apparatus comprising
(a) a signal line connected to a signal electrode; and
(b) a reference line connected to a reference electrode;
   said signal line and reference line being associated by being in close physical proximity for a substantial part of their lengths, said electronic apparatus further comprising subtraction means for subtracting an interference signal on the reference line from an interference signal on the signal line thereby to enhance a desired signal on the signal line.

A second aspect of the present invention provides an electronic apparatus for reducing interference in a desired signal, the apparatus comprising:
(a) a plurality of signal lines, each connected to a respective signal electrode; and
(b) a plurality of reference lines, each connected to one or more counter electrodes;
   each of said signal lines being associated by being in close physical proximity with a respective one of said reference lines for a substantial part of their lengths, so that each signal line with its corresponding reference line forms a signal line/reference line pair, said electronic apparatus further comprising subtraction means for subtracting an interference on each reference line from an interference signal on the associated signal line in that signal line/reference line pair.

A third aspect of the present invention provides a method of reducing interference from a desired signal, the method comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the interference signal on the reference line from the interference signal on the signal line.

A fourth aspect of the present invention provides a method of reducing interference from a desired signal, the method comprising
(a) providing a plurality of signal lines, each carrying a desired signal and an interference signal;
(b) providing a plurality of reference lines, each carrying at least an interference signal, each signal line being associated by being in close physical proximity for a substantial part of its length with a respective reference line to provide respective signal line/reference line pairs; and
(c) performing a subtraction step of subtracting the interference signal on each respective reference line from the interference signal on the associated signal line of its reference signal line/reference line pair.

A fifth aspect of the present invention now provides an electronic apparatus for reducing interference in a signal derived from an EPM the apparatus comprising
(a) a signal line connected to a signal electrode;
(b) a reference line connected to a reference electrode; and
(c) at least one ground line for said signal line and reference line, said ground line or lines being connected to at least one ground electrode or individually to respective ground electrodes;
   said electronic apparatus further comprising subtraction means for subtracting an interference signal on the reference line from a signal on the signal line.

A sixth aspect of the present invention provides an electronic apparatus for reducing interference in a desired signal, the apparatus comprising:
(a) a plurality of signal lines, each connected to a respective signal electrode; and
(b) one or more reference lines connected to one or more reference electrodes; and;
(c) one or more ground lines connected to one or more ground electrodes;
   said electronic apparatus further comprising subtraction means for subtracting an interference on the or each reference line from an interference signal on the signal lines and/or subtracting an interference signal on the or each ground line from the interference signal on the signal lines.

A seventh aspect of the present invention provides a method of reducing interference on a signal derived from an EPM, the method comprising
(a) providing a signal line carrying a desired signal and a first interference signal, said signal line being connected to a signal electrode;
(b) providing a reference line carrying at least a second interference signal, said reference line being connected to a reference electrode;
(c) providing a ground line for said signal line and reference line, said ground line or lines being-connected to at least one ground electrode or individually to respective ground electrodes; and
(d) a subtraction step of subtracting the second interference signal on the reference line from the first interference signal on the signal line.

An eighth aspect of the present invention provides a method of reducing interference from a desired signal, the method comprising
(a) providing a plurality of signal lines, each carrying a desired signal and a first interference signal;
(b) providing one or more reference lines carrying at least a second interference signal;
(c) providing one or more ground lines; and
(d) performing a subtraction step of subtracting the second interference signal from said first interference signal.

A ninth aspect of the present invention provides an electrode support structure apparatus for effecting an EPM, the apparatus comprising an electrode support having supported thereon, an array of signal electrodes presented for contacting the skin of a subject, first connection means being provided for independent electrical connection to each of said signal electrodes, the apparatus further comprising an electrically insulated mesh having one or more of reference nodes and second connection means for independent electrical connection to the or each of said reference nodes.

A tenth aspect of the present invention provides a combined fMRI and EPM apparatus comprising an MRI unit and an EPM measurement system which comprises an electronic apparatus according to any of the first, second, fifth, sixth or ninth aspects of the present invention.

According to a eleventh aspect of the present invention, there is provided a method of performing a simultaneous fMRI and EPM measurement, the method comprising placing a subject in an MRI apparatus and performing MRI scans and simultaneously taking EPM measurements by a method which comprises a method of reducing interference in accordance with any of the third, fourth, seventh or eighth aspects of the present invention.

Any apparatus according to any of the first, second, fifth, sixth, ninth or tenth aspects of the present invention optionally may comprise in addition, any essential, preferred or exemplary feature of any other of these, unless already essential for the apparatus of that aspect of the invention.

Thus, the totality of the disclosure or this patent specification includes the subject matter of any dependent claim appended hereto in combination with the subject matter of any claim or claims on which it is dependent but optionally, also in combination with the additional subject matter of any claim on which it is not dependent.

Any method according to any of the third, fourth, seventh, eighth or tenth aspects of the present invention optionally may comprise in addition, any essential, preferred or exemplary feature of any of these, unless already essential for the method of that aspect of the invention.

Any method according to any aspect of the present invention may also include the method when any or all signal, reference, reference and ground lines are electrically connected to their respective electrodes as defined in accordance with the corresponding electronic apparatus according to the present invention.

### DETAILED DESCRIPTION OF THE INVENTION

A “reference loop” is used for subtracting interference voltages induced by external alternating magnetic fields induced into the circuit loop formed by the connection between the living body and electronic amplification circuitry. In the described embodiments, a simplified version of the reference loop is described for use in multi-channel EPM recordings, such as EEG recordings in order to reduce noise voltages induced by the magnetic fields generated in a functional magnetic resonance imaging device (fMRI). In addition, an embodiment of a complete circuit means is described for acquiring simultaneous EPM in the fMRI environment, with minimal interference to the EPM and fMRI.

In order to achieve EPM data acquisition, concurrent with fMRI, the EPM data acquisition circuitry must reject interference caused by external (to the body) electric and magnetic fields. The main sources of interference are low frequency electric and magnetic fields from the AC power mains (commonly 50 or 60 Hz), switched magnetic fields from fMRI with fundamental frequencies ranging down to approximately 1 kHz, and radio frequency (rf) electromagnetic fields from fMRI ranging from 60 to 130 MHz. In addition, the large static magnetic field of the MRI scanner causes interference voltage to be induced in EPM signal lines whenever movement of the electrodes or lead wires occurs.

In the broadest aspect, the present invention utilises a single signal line and a single reference line. However, most practical applications will involve use of a plurality of signal lines with associated reference lines. The single signal line can be connected to a respective separate signal electrode. The reference lines may be connected to a single reference electrode or to a respective separate reference electrode or any other arrangement involving multiple reference electrodes.

Each signal line may therefore be associated with a corresponding one of the reference lines to be in close proximity for a substantial part of their lengths, so that each respective signal line and associated reference line constitutes a respective signal line/reference line pair. The subtraction means is then arranged to subtract an interference signal on each reference line from the interference signal on its associated signal line in the pair, thereby enhancing the desired signal on that signal line.

Any reference line is preferably connected to a conductive member physically close to, but not in direct electrical contact with the part of the human body (eg the scalp in the case of an EEG measurement). This conductive member, may for example, be in the form of a conductive mesh. In other embodiments, the reference lines may be in direct electrical connection with the subject, eg in the case of EEG to a signal electrode which may, for example be in contact with an earlobe or with skin close to an ear.

Essential for some, whilst merely preferable for other aspects of the present invention is provision of one or more ground lines. Any signal line/reference line pair may share a common ground line, preferably in close physical proximity with both, or each signal line and reference line may be provided with its own ground line, preferably in close physical proximity therewith. A combination of such arrangements is also possible (one or more shared ground lines for some signal/reference line pairs and one or more individual ground lines for any one or more others). All ground lines may be connected to a common ground electrode or to individual respective ground electrodes, or any other arrangements involving multiple ground electrodes. Preferably, the or each ground electrode is in direct (low resistance) contact with the subject (eg the skin of the head or scalp in the case of EEG), as described further hereinbelow.

Where an individual line or lines (signal, reference or ground) is or are connected to its, or their, own dedicated electrode (signal, reference, or ground, respectively), that electrode may be embodied as two or more electrode entities with the reference line or lines being connected thereto in parallel. The terms “electrode” and “node” (see below) are to be interpreted as encompassing these possibilities, except where explicitly stated to the contrary or where the context forbids.

When both a reference line and also a ground line are employed, then the or each signal line may be in close physical proximity for a substantial part of the length thereof, with a respective reference line, a respective ground line, or both.

Preferably, signal and any ground electrodes are in direct electrical connection with the subject (usually the head, or head/neck region when the EPM is EEG, e.g. mainly to the scalp). This preferably means an individual electrode contact resistance of less than 1 Kohms. However, reference electrodes are preferably not in direct electrical contact with the subject but are electrodes in close physical proximity with the subject, preferably each respectively close to its associated signal electrode. Thus, the resistance between any reference electrode and the subject is preferably 1 Kohm or more, especially 10 Kohm or more.

Preferably, and particularly when the EPM is EEG the reference electrodes are arranged as a mesh. Then signal and reference electrodes may be arranged over the head or scalp but one signal/reference electrode pair may be attached to positions where the pick-up of physiological electrical signals will be low, such as beneath the ear. Thus, it is to be understood that the term “electrode” includes variants which are not in direct electrical contact with the subject.

A preferred form of construction comprises a flexible, elastic reference mesh material acting as a cap to hold the electrodes in place. The reference mesh material may be coated with an insulating layer to electrically isolate the mesh from the body and electrodes. All components are preferably made from materials chosen to be resistant to chemical disinfectants.

As used herein, any electrical contact point to a reference mesh is usually termed an “electrode”. However, the term “node” is also used for such a contact point with a reference mesh and as such, can be considered synonymous with electrode, whether or not any part of the mesh is in direct electrical contact with the subject, eg with the skin of the subject. One suitable form of construction is in the form of a cap, preferably having two layers of insulating elastic cap material with a reference mesh construction sandwiched between, and electrodes anchored to the cap. Cap structures for supporting EEG electrodes are already known from WO-A-00/27279 and U.S. Pat. No. 6,708,051.

Each electrode site on any suitable cap structure, preferably has four wires—two for the signal loop and two for the reference loop—arriving as two twisted pairs twisted around each other. One wire connects to the body electrode; one wire connects to the reference mesh next to the electrode; one wire proceeds across the cap to the body ground electrode; and one wire proceeds across the cap to the reference mesh ground connection. A multi-channel arrangement would comprise a plurality (n) of such sites.

Reference mesh material can be made of carbon loaded fabrics, foam or yarns (carbon wire). Other conductive materials can be used for loading in addition to or in lieu of carbon.

For the avoidance of doubt, reference to subtraction in accordance with the present invention means any attenuation of interference on a signal line by deriving an interference signal from a corresponding reference line and using it to diminish the interference signal on the signal line. Arithmetic subtraction as well as other operations are included within this term. The definition includes substantial total elimination of the interference signal but also covers at least some diminution of the interference signal from the signal line.

Reference herein to any two or more lines being associated in close proximity for a substantial part of their length(s) preferably means that the perspective lines run in close physical proximity for at least 50%, more preferably at least 60%, still more preferably at least 70%, yet more preferably still at least 80% and most preferably at least 90% of their lengths (when one or more wires is longer than any other relevant wire, then these percentages are of the longest).

Any lines which are in close proximity may be arranged thus by any suitable means, eg coaxially (such as with the reference line surrounding a core of the signal line, or vice versa) or by being run together as a twin wire pair (or multi-wire group) or by any other means, but most preferably, by being twisted together.

The subtraction means preferably comprises a differential amplifier with inverting and non-inverting inputs connected to signal line(s) and reference line(s) respectively.

Each signal line/reference line pair may be shielded, for example by a metallic sheathing which suitably may be connected to a ground connection.

The subtraction means preferably also comprises one or more common mode chokes associated with the respective signal line/reference line pairs, the windings of each such common mode choke being connected to a respective one of the signal line and the reference line. The subtraction means preferably also comprises low pass filter means, especially a second order low pass filter, an exemplary embodiment of which comprises a Bessel type filter.

The apparatus and method of any aspect of the present invention may be deployed in the MRI room itself. The apparatus of any aspect of the present invention may be substantially totally electrically wired, ie not require any optical or wireless link, although the latter are also possible.

The present invention will now be explained in more detail by way of the following description of preferred embodiments, and with reference to the accompanying drawings, in which:

### DESCRIPTION OF THE DRAWINGS

FIG. 1 shows a schematic of an EEG and fMRI set up, in which an interference reduction apparatus according to the present invention may be employed;

FIG. 2 shows the fMRI pulse sequence employed in the set-up of FIG. 1;

FIG. 3 shows a circuit diagram of a first embodiment of an electronic interference reduction apparatus according to the present invention;

FIG. 4 shows a circuit diagram of a second embodiment of an electronic interference reduction apparatus according to the present invention;

FIG. 5 shows a circuit diagram of the second embodiment of FIG. 4;

FIG. 6 shows an EEG plot obtained during tests utilising the arrangement of FIG. 1 in combination with an electronic interference reduction apparatus according to the present invention;

FIG. 7 shows an equivalent circuit of an embodiment utilising both a ground electrode and a reference electrode;

FIG. 8 shows an equivalent circuit of input circuitry suitable for use with an arrangement such as shown in FIG. 7;

FIG. 9 shows an equivalent circuit arrangement of interconnection of multiple signal electrodes on the body together with a ground electrode;

FIG. 10 shows suitable amplification, subtraction and filtering circuitry for use with arrangements generally as depicted in FIGS. 7 to 9;

FIG. 11 shows front-end circuitry forming part of a particularly preferred embodiment of the present invention which utilises reference electrodes and ground electrodes;

FIGS. 12 and 13 show side views of EEG electrode connections to the human head for use in the embodiment which comprises the circuitry shown in FIG. 11;

FIG. 14 shows the arrangement of scan head and circuitry for the embodiment of FIGS. 11-13, with respect to the shielded scanner room;

FIGS. 15 and 16 show intermediate circuitry inside a shielded amplifier enclosure, which receives signals from the front-end circuitry shown in FIG. 11; and

FIG. 17 shows the location of the circuitry of FIGS. 15 and 16 within the shielded amplifier enclosure, relative to the shielded scanner room and exterior control room.

### DESCRIPTION OF PREFERRED EMBODIMENTS

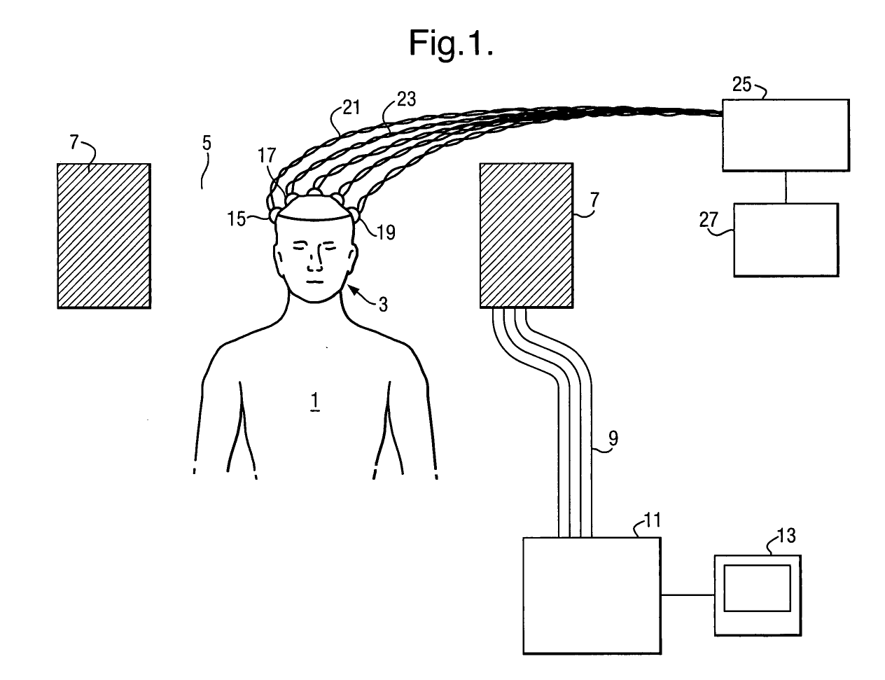

FIG. 1 shows a basic fMRI and EEG system in which the apparatus and method of the present invention may be employed.

As shown in FIG. 1, a subject 1 is arranged with the subject's head 3 located within the bore 5 of an fMRI coil unit 7 which carries the magnetic field windings and rf coils. These coils and windings are energised via a multiplicity of wiring connections 9 etc which connect the coil unit 7 to operational circuitry 11. The operational circuitry unit is connected to a memory and display unit 13 whereby the MRI scans can be stored, displayed and printed at will.

A plurality of electrodes 15, 17 etc for obtaining EEG signals are attached to the scalp of the subject 1. As will be explained in more detail hereinbelow, one of these electrodes 19 is a “reference electrode”. Signals from the electrodes 15, 17, 19 etc are conveyed by wires 21, 23 etc to an EEG control unit 25 which is connected to a recorder 27.

The combined fMRI/EEG arrangement may be considered to apply to any specific embodiment of EEG processing circuitry described hereinbelow.

In a worked embodiment, used for obtaining data presented in more detail hereinbelow, the MRI system was the Siemens Allegra™ (3.0 T)-MR6.

The Siemens Allegra™ 3T is a head-only research magnet. It has the necessary hardware and software to perform basic and clinical scans. Gradient hardware consists of a 36 cm I.D. asymmetric gradient coil capable of imaging at 60 mT/m with slew rates in excess of 600 T/m/s at a duty cycle of 70% allowing single shot echoplanar imaging ( EPI) at a sustained rate of 14 images/second. The system has a 15 kW RF amplifier, and 8 RF preamp channels, This system supports the Syngo™ software on a Windows™ NT platform.

The EPI regime employed 1 to 8 gradient switching pulses (images) per second. Gradient strength: 25-35 mT/m, max 40 mT/m; Slew rate: 400 mT/m/msec. Pulse width: 0.32-0.64 msec, oscillating between positive and negative gradients. Rf pulse freq: 126 MHz, frequency modulated for slice position.

The conventional sequence used for fMRI is multi-slice echo planar imaging. In this, the largest gradient is applied as a bi-polar square wave, which is often modified to be more trapezoidal or sinusoidal in form (to smooth the edges). Typically for one image this is applied for 20-100 ms with a fundamental frequency of 2 to 0.5 kHz. One of the other two gradients is usually applied as a series of smaller pulses (100 μs duration typical) at the zero crossings of the big switched gradient, whilst the third (slice select) gradient is generally just applied at the beginning of the sequence as a bi-polar square pulse, typically lasting 3-5 ms. The rf is usually just applied at the same time as the slice select gradient.

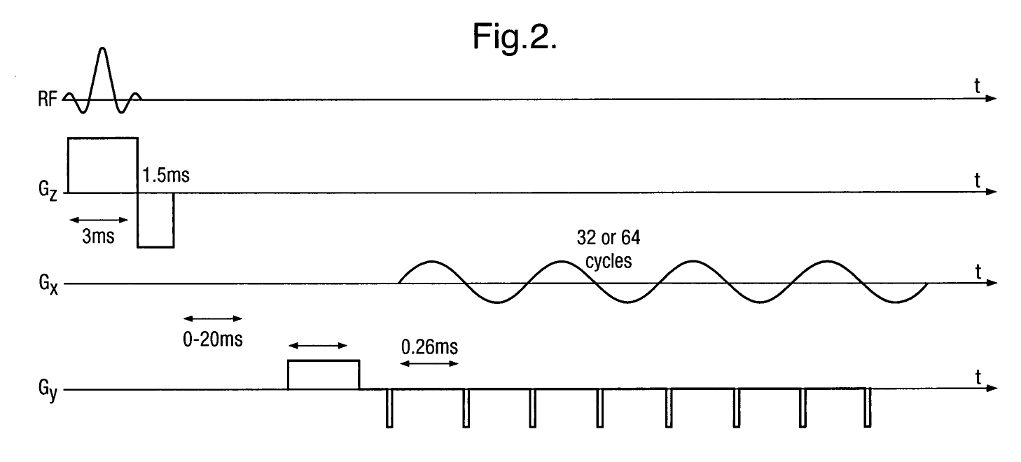

FIG. 2 shows the EPI sequence used. Gz denotes slice select, Gx is the large gradient and Gy is the smaller pulsed gradient. The rf pulses are also shown. In the tests described further hereinbelow, Gx was on for 30 ms. Depending on MRI machine used, slice gradient times can vary by a factor of 2, and the switched gradient could be lower by a factor of 2 in frequency and strength.

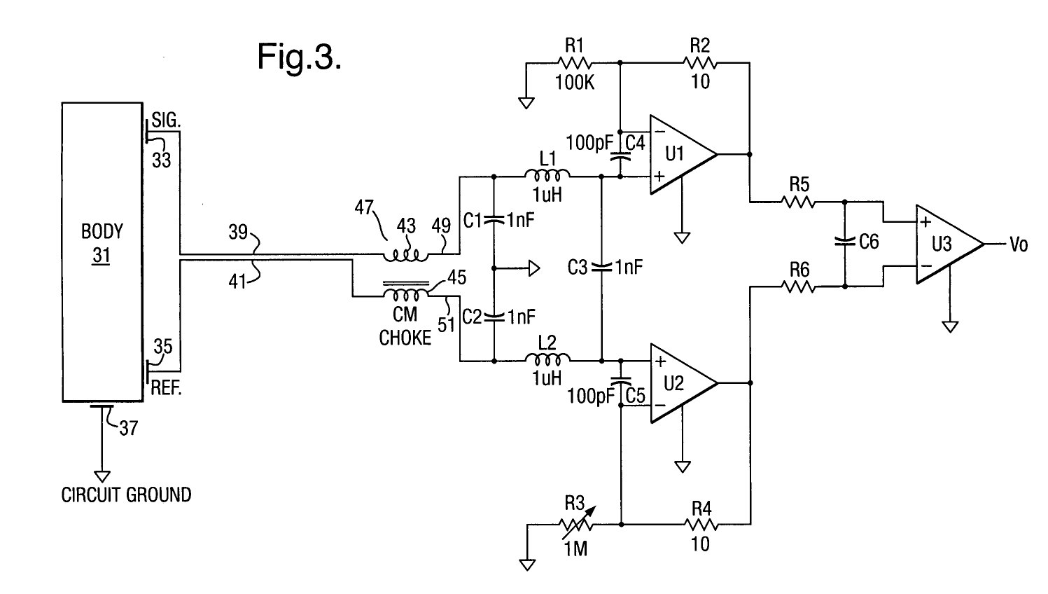

FIG. 3 of the accompanying drawings shows a singe channel of EEG data acquisition circuitry of a first embodiment of the invention. It incorporates a reference loop and other means for suppressing interference generated by fMRI. As shown in this figure, attached to a head of as subject 31 are a signal electrode 33, a reference electrode 35 and a ground electrode 37 for biopotential signal acquisition. In order to minimize rf noise in the EEG signal, the electrodes are not metallic, but preferably carbon-loaded material. In order to minimize interference to fMRI, the use of metals, glues, epoxies, etc. should be avoided.

Wires 39 and 41 respectively run from the signal electrode 33 and reference electrode 35 and are physically placed as close together as possible. Since the electrode lead wires 39, 41, 42 are made of carbon fiber, wire electrodes can be implemented simply by using the ends of the wires held in place mechanically on the scalp or earlobe and electrically connected to the body with electrode gel. The reference electrode 35 is preferably located on an earlobe, and its wire 41 is run directly to signal electrode 33 located on the scalp. Wire 39 connected to sig electrode 33 is twisted with wire 41 and the twisted pair, of length approximately from 2 to 5 meters is connected to filtering and amplification circuitry as described further hereinbelow. In multi-channel applications comprising a plurality of signal electrodes, each signal electrode wire is paired with a separate wire coming from the reference electrode and all the shielded twisted pairs are bundled together with the ground reference lead wire to form the electrode cable set.

The “reference loop” is the circuit formed by following the path from the reference electrode 35 through its associated wire 41, into a non-inverting input of amplifier U2, then to circuit ground leading back to the body through ground electrode 37. An analogous loop is formed in the signal pathway from signal electrode 33 through wire 39 into the non-inverting input of a first amplifier U1 and back to the body through circuit ground and ground electrode 37.

The large magnetic fields of fMRI induce voltages on the order or volts in these loops. The induced voltages are reduced by minimizing the areas of the loops, but the physical arrangement of electrodes on the scalp versus the site of the ground electrode results in a loop that cannot be avoided and is large enough to result in large induced voltages. Since the reference voltage is subtracted from the signal voltage (a standard practice used to reduce interference in EEG), spatially associating the signal and reference loops in close proximity results in further reduction of the induced interference. However, standard practice is to use a single wire from the reference electrode for all of the signal channels, which results in large spatial mismatching for most channels. In practice, a plurality of signal electrodes, each with its own signal wire will usually be used. A separate reference wire will then be employed for each signal channel, closely following the signal lead wire (preferably twisting the wires) so that spatial matching of the loops is maximized. All of the reference wires terminate electrically at the reference electrode or reference electrodes, if more than one of the latter is provided. This means that in such an arrangement, many reference wires terminate on a single reference electrode or group of reference electrodes.

Returning to FIG. 3, at their respective ends remote from the signal 33 and reference 35 electrodes, shielded twisted pair wires 39, 41 are connected to respective windings 43, 45 of a common mode choke 47. Common mode (voltages that are the same for both wires) rf is greatly reduced by the common mode choke 47 in combination with capacitors C1 and C2, having their mutual connection point connected to ground, in series between the output terminals 49, 51 of the choke winding 43, 45 remote from the connections of the twisted pair wires 39, 41. Residual differential mode rf is thereby converted to common mode by inductors L1 and L2 respectively connected at one end to choke outputs 49, 51 and at their other ends, bridged by a third capacitor C3. The inductors L1 L2 are typically 1 pH but ferrite beads having an impedance of several hundred ohms at the relevant rf frequency may be located on the wires. These should be situated sufficiently from the static magnetic field of the scan head to avoid saturation. Capacitors C1, C2 and C3 must be small (approximately 1 nF) in order to maintain a high impedance for low frequency signals coming from the electrodes.

First and second low noise operational amplifiers U1 and U2 have a high input impedance with gains approximating one and respectively receive signals at their non-inverting inputs from inductors L1 and L2. Amplifiers U1 and U2 serve as impedance transformers, presenting a high impedance to the electrodes and a low impedance driving the respective inverting and non-inverting inputs of a third amplifier U3. The gains of amplifiers U1 and U2 are set by resistors R1 to R4, with R3 being variable to match closely the gains of U1 and U2. Capacitors C4 and C5 are connected between the respective inverting and non-inverting inputs of U1 and U2 to minimise the low frequency response of the amplifiers caused by rectification of any remaining rf appearing at the inputs. The outputs of U1 and U2 are connected to resistors R5 and R6 respectively in series with with respective inverting and non-inverting inputs of a third operational amplifier U3. These combine with a capacitor C6 (across the inputs of U3) to convert differential mode voltages above a set −3 dB (filter cutoff) frequency to common mode voltages. U3 is a high speed differential amplifier (such as Analog Devices™ AD 8129) that is capable of rejecting common mode voltages up to rf.

Thus in combination, R5, R6, C6 and U3 function as a single pole low pass filter, converting differential mode voltages on the signal and reference lines to common mode voltages on a −6 dB per decade basis above the −3 dB cutoff frequency. Such a filter converting differential mode voltages to common mode voltages is hereinafter referred to as a DM/CM filter). U3 also performs subtraction of the reference voltage from the signal voltage in the bandwidths below the DM/CM filter cutoff frequency. Any mismatch between interference voltages in the signal and reference lines below the DM/CM cutoff frequency, results in a residual interference component in the signals. Above the cutoff frequency, both signal and reference signals are filtered but since the filter is only single pole, any large mismatches in noise voltage appearing on the signal and reference lines at frequencies near the filter cutoff, will result in residual interference appearing at the output.

In this embodiment, the DM/CM filter cutoff frequency is set as low as possible to obtain maximum rejection of magnetically induced interference voltages. Typically, R5 and R6 may be 365 Ω and C6 may be 1.0 pF, resulting in a −3 dB cutoff frequency of approximately 218 Hz. U3 amplifies the remaining differential mode signal received from U1 and U2 by a gain of 10, and this output is further amplified and filtered with high pass and low pass filters (not shown). A typical filter implementation includes a single pole high pass filter with a −3 dB frequency of 1.0 Hz and a 4-pole Butterworth low pass filter with −3 dB frequency of 256 Hz. The combination of all filters results in a final signal bandwidth, which in this embodiment is from 1 to 100 Hz. To reduce interference still further, the bandwidth may be narrowed, depending upon the frequency range of the signal of interest.

Benefits of circuit are provided by the use of separate wires from the reference electrode for each signal wire (reference loops), the common mode choke, and the combination of gain-matched buffer amplifiers, DM/CM filter and a high speed differential amplifier. Using the ends of the carbon lead wires as electrodes and the use of a second shield with a hybrid ground connection is also advantageous.

The fundamental object of the circuit shown in FIG. 3, is to reduce interference voltages to low levels and amplify the signal. This has to be achieved across the wide range of frequencies involved. For attenuation of power mains interference, the high impedance of the buffer amplifiers, tight gain matching, the high common mode rejection of U3, tight matching of reference loops and isolated amplifier ground are sufficiently effective. Driving the twisted-pair shield maintains a high input impedance when using long electrode leads, especially when the wires are within a shield connected to the amplifier ground. For interference from fMRI magnetic fields, tightly matched reference loops significantly reduce induced voltages, and the R5-R6-C6-U3, DM/CM low pass filter in combination with the 4-pole low pass filter removes most remaining interference. The use of carbon wires, the second shield with hybrid ground, rf common mode and differential mode filters, rf shunt capacitors across the buffer amplifier inputs and the high speed differential amplifier also act in combination to eliminate rf interference.

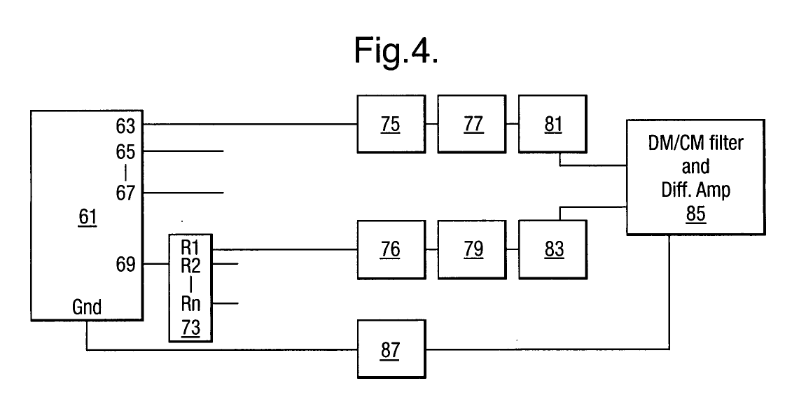

Another circuit embodiment according to the present invention is depicted in FIG. 4, in which numeral 61 represents the subject, with signal electrodes 63, 65, 67 etc (typically attached to the scalp), reference electrode 69 (typically attached to the earlobe), and common electrode 71. The electrodes and connecting wires are typically carbon loaded material (to lower conductivity thus reducing rf currents in the electrodes and wires), with a 10 K Ω) to 15K Ω carbon resistor (not shown) inserted in line near the electrode for rf current-limiting safety and filtering. Numeral 73 represents a conductive junction (typically carbon loaded material for rf current reduction) for distributing a multitude of reference wires (also carbon loaded material) each of which is placed in close proximity to, and where possible, twisted with, a signal electrode wire. Each reference wire forms a reference loop that is closely matched to the loop formed by the signal electrode wire.

The signal and reference wire pairs are bundled together along with the ground electrode wire for about from 2 to 5 meters typically, at which point the carbon wires are terminated inside a shielded metal (aluminum) enclosure containing rf filters 75 etc for each wire (only one is shown for electrode 63 for simplicity in the diagram). The metal case of the rf filter enclosure is bonded to the frame of an MRI apparatus for establishing a low impedance rf ground. The rf filters consist of series inductors (typically 1 μH) followed by a capacitance connected to an isolated rf ground in the enclosure, which is connected, in turn, to the metal case by a single 1 nF capacitor. Metallic (usually copper) wires in twisted pairs are connected to the outputs of the rf filters for each signal-reference pair, and a single metal wire is connected to the ground electrode rf filter output, with the resulting cable bundled inside a metallic shield (shield connected to ground at the rf filter box). This cable is run (typically 2 meters) to a metallic (aluminum) enclosure containing preamplifiers, filters, differential amplifiers, filters, main amplifiers, sample-and-holds, digitizer, digital control and ethernet interface circuitry. The shield of the cable from the rf filter box terminates on the metal casing of the amplifier/digitizer enclosure.

Each signal-reference wire pair 63/69 etc is connected via the rf filters 75, 76 etc to a respective pair of preamplifiers 77, 79 etc. At the input of the preamplifiers 77, 79 etc, additional rf filtering is implemented by using a common mode choke across the wire pairs followed by capacitors to isolated ground, and a series indicator (1 μH typically) or a ferrite chip (presenting an rf impedance of several hundred ohms) in each line followed by a capacitor (typically 1 nF) across the lines as in the embodiment of FIG. 3. An rf filter 87 consisting of a series inductor (1 μH typically, or a ferrite chip) followed by a capacitor to ground (1 nF typically) is also placed in the ground line. The output of each preamplifier is connected to a low pass filter. These are denoted as low pass filters 81, 83 etc for the preamplifier par 72, 79. Thus, each signal line from a signal electrode 63 and each reference line associated therewith is connected to its own rf filter, with the outputs of the low pass filters connected to a circuit unit (denoted as DM/CM filter and Diff Amp 85). The circuit unit performs filtering and subtracting functions in a manner similar to that of the embodiment of FIG. 3.

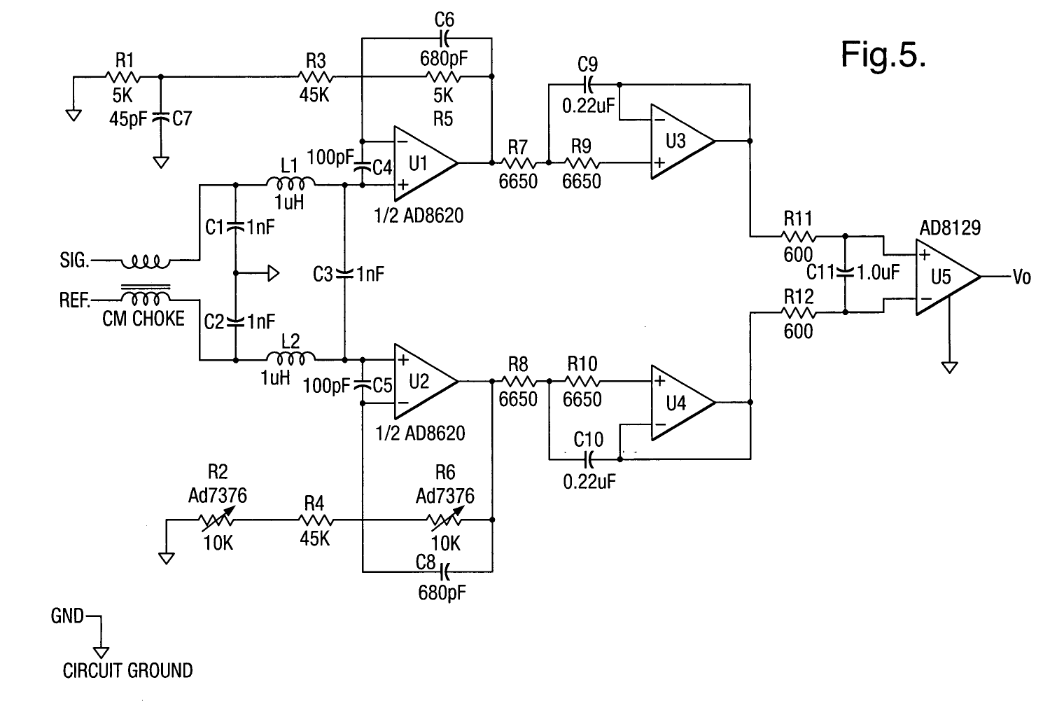

Referring to FIG. 5, the preamplifiers for a signal reference pair are preferably Bi-FET, JFET or CMOS operational amplifiers U1 and U2, with low noise and high input impedance. U1 and U2 may be implemented in the form of a dual operation amplifier integrated circuit such as Analog Devices AD8620 or OP2177. A capacitor (C4 and C5, typically 100 pF) may be connected across the inverting and non-inverting inputs of the operational amplifiers to minimize low frequency response at the op amp output caused by rectification of residual rf at the inputs.

The preamplifiers have a gain of approximately 1 to 2, and serve primarily as impedance transformers to compensate for the relatively high impedance of the electrode-tissue interface. Each signal preamplifier U1 has a fixed gain, while the reference preamp may have a variable gain (adjusted by varying feedback resistance around the operational amplifier using digitally controlled resistors R2 and R6), which allows dynamic trimming of the reference voltage amplitude, to provide a better match of interference voltages on the signal and reference lines for subsequent subtraction. R2 and R6 may be implemented with Analog Devices AD7376 digital potentiometers with 10 K Ω resistance. In the circuit implementation shown in FIG. 5, the gain of the signal preamplifier is 1.1 and the reference preamplifier gain varies from 1.0 to 1.2. Wider ranges may be used by setting the gain of the signal preamplifier to the centre of the range (for example, center gain of 2.0) and varying the reference preamplifier gain between the edges of the range (for example, a range of from 1.0 to 4.0).

Since the digital potentiometers present a capacitance in addition to a resistance to the preamplifier feedback circuit, compensation capacitors C6 and C7 (typically 680 pF for the AD7376) are added to the feedback loops of the preamplifiers. C7 (typically 45 pF) is used in the signal preamplifier feedback network as shown, to match a capacitance added by R2 in the reference preamplifier feedback network, in order to maintain similar frequency responses for the preamplifiers.

A second order low pass filter 81, 83 etc (preferably of the Bessel type to minimize pulse overshoot) with a gain of one follows each preamplifer. As shown in FIG. 5, operational amplifiers U3, U4 (AD 8620, OP 2177 or similar) and circuit elements R7-R10 and C11-C12, construct second order Bessel filters with a cutoff frequency of 145.4 Hz. The resulting filtered signal and reference voltages are input via a first order DM/CM filter, to wide bandwidth differential amplifier U5 (with a typical gain of about 10). For the purpose of filtering both signal and reference lines, and also, subtracting the reference from the signal in the bandwidth below the filter cutoff frequency, similar to as in the first embodiment. However, with correct selection of cutoff frequencies for the Bessel and DM/CM filters, a third order low pass filter may be realised at the output of the differential amplifier, instead of a single order filter as in the first embodiment. Thus, better filtering of the interference is achieved. In FIG. 5, circuit elements R1, R12 and C13 in combination with U5 (Analog Devices AD 8129 or similar) form a DM/CM filter with a −3 dB frequency of 132.8 Hz. The resulting third order filter has a −3 dB cutoff frequency of 100 Hz. Following the differential amplifier, additional stages of amplification and low pass filtering are employed, as usually practiced in the acquisition of EEG. The ground electrode lead (after r.f. filtering) is connected to isolated circuit common node. Isolation is held to approximately 1 nF in order to allow low impedance for rf filtering yet maintain high impedance for low frequency interference rejection and patient safety.

An interference circuit according to the present invention was tested with a Siemens Allegra™ commercial fMRI machine as hereinbefore described, using a live subject. Three channels of EEG were implemented using locations on the scalp with reference on an earlobe and ground on the back of the neck. All leads were carbon wires with 10K Ω carbon film resistors inserted close to the electrode sites. The ends of carbon wires served as electrodes placed in a small amount of electrode gel to lower the impedance to scalp and skin. Each signal lead was twisted with a parallel reference lead as described in the foregoing embodiments.

EEG hardware consisted of a three channel preamplifier with rf and interference filtering. Rf filtering was implemented as described in the first embodiment. The preamplifiers and filters were implemented as described in the second embodiment, with variations. Channel 1 had a variable gain reference preamplifier, implemented with digitally controlled resistors, whilst channels 2 and 3 had voltage followers on both signal and reference lines. All channels had a 2-pole 145.4 Hz Bessel filters in stage 2, combined with 1-pole 132.8 Hz DM/CM filters on the differential amplifier inputs, to form 3-pole low pass filters. With additional stages of amplification and filtering, the resulting −3 dB signal bandwidth was 1-95 Hz and the sample rate for digital conversion was 250 Hz.

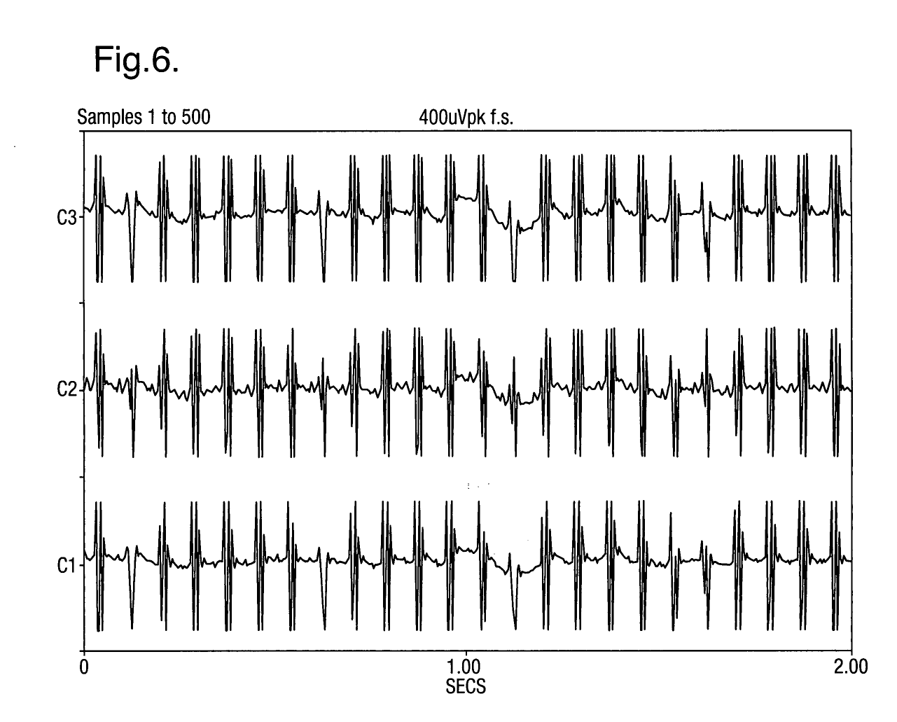

FIG. 6 shows an EEG taken for 2 seconds during EPI scanning. Interference from rf and switch magnetic fields generated by the fMRI scan was reduced by the interference circuit to approximately 300 μV, referred to the input. This was well within the dynamic range of the amplifiers, 400 μV, thus allowing the amplifiers to avoid saturation and recover rapidly between pulses. In addition, the residual interference is low enough to be digitally filtered without resorting to high sample rates, thus resulting in substantially complete elimination of the fMRI noise.

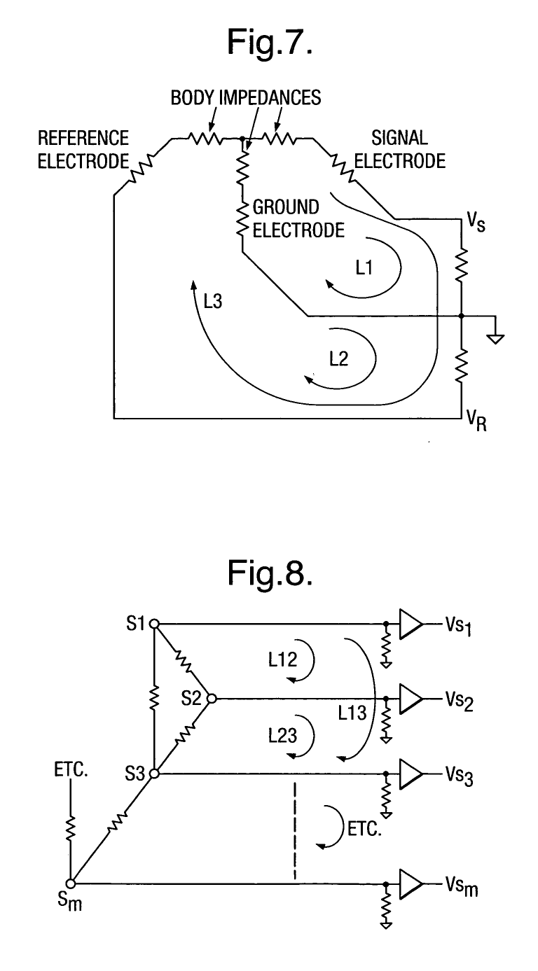

FIG. 7 shows an equivalent circuit for a single channel reference loop arrangement as shown and described in respect of the earlier embodiments. However, in this class of embodiments, there are three loops formed by the circuits that comprise the signal, reference and ground electrodes and associated wires and impedances.

External varying magnetic fields passing through the area formed by the loops could induce unwanted voltages in the circuit which obscure the desired signal voltages detected on the body. However, the interference voltages are reduced by minimizing the area formed by the loops, and may also be reduced by subtracting the voltage appearing on the reference circuit from the voltage on the signal circuit, since with appropriate spatial arrangement, there should be no physiological signal of interest in the reference circuit. In the equivalent circuit of FIG. 7, if the areas formed by loops L1 and L2 are well-matched, subtracting the reference voltage Vr from signal voltage Vs will significantly reduce or cancel the magnetically induced interference induced in the signal channel due to loop L1. However, a third loop L3 is formed via the low impedance of the body and electrodes, since the reference loop is connected to an earlobe.

The interference induced in loop L3 may be minimized by reducing the loop area. Alternatively, loop L3 may effectively be eliminated by removing the reference lead from connection to the earlobe, with a separate ground return added to complete the circuit for loop L2.

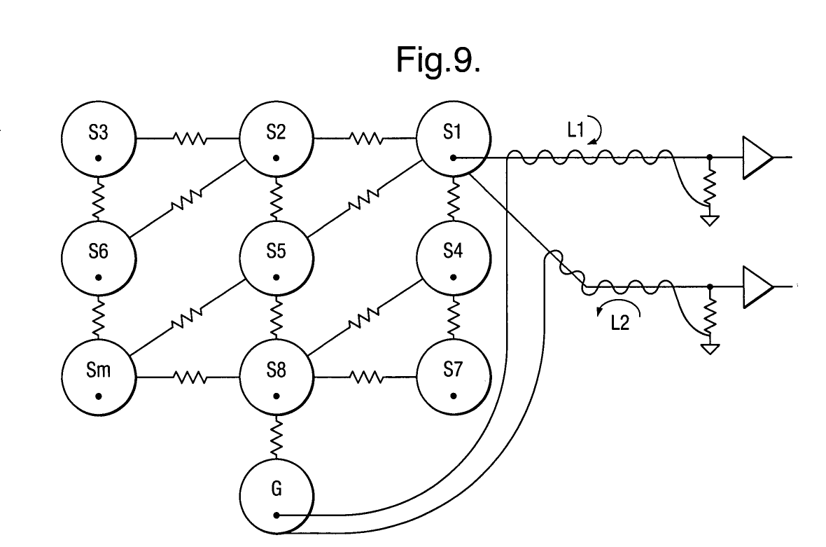

FIG. 8 shows an equivalent circuit for demonstrating another source of interference which may not be so well reduced by the circuit arrangements of the embodiments of FIGS. 1-5. As depicted, all signal leads (S1, S2, . . . Sn) are connected via the impedances of the electrodes and body (shown as single resistors between various signal electrode sites), thus forming loops. A parallel pathway for the reference loop circuit that is well-matched to each signal-signal loop is required in order to cancel the magnetically induced interference by subtraction of the reference loop voltage. The embodiments of FIGS. 1-5 effectively only provide a single reference loop for each signal channel, but that reference loop does not match the additional loops formed by the multitude of signal channels, as shown in FIG. 8.

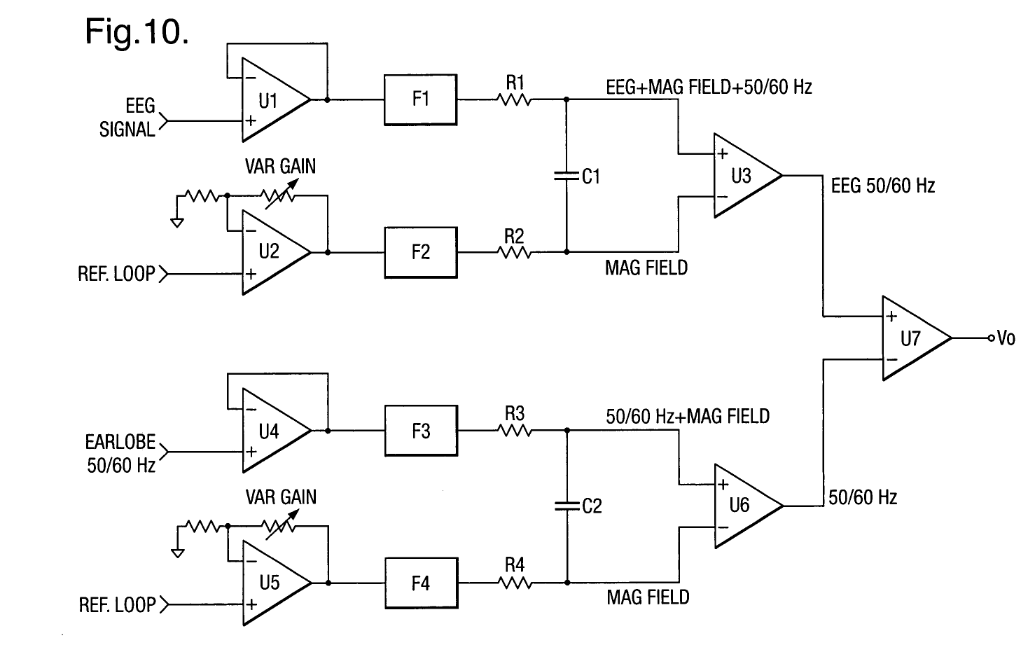

To better match the totality of loops in each signal channel, another embodiment of the invention employs an isolated reference network or mesh. A section of multiple electrodes mounted on the body with accompanying reference loop network or mesh is shown in the equivalent circuit of FIG. 9, with a single channel comprising signal and reference circuits as shown. For clarity, only the pathways formed by the reference mesh are shown (as resistors connected to rings around each signal electrode) with the signal electrode site designated by a dot located within the ring in an area cleared of the reference mesh. Each signal site is assumed to be connected to all other signal sites (and ground electrodes designated by “G”) via electrode and body impedances as depicted in FIG. 8, but not shown in FIG. 9.

As can be seen in FIG. 9, any pathway between signal electrodes is closely matched by a reference pathway formed by the conductive reference network. To obtain the best match of induced voltages in the loops, the impedances of the pathways in the signal and reference loops should be similar. The loops L1 and L2 of the equivalent circuit of FIG. 7 are thus physically well matched and smaller in area since each signal and reference circuit has a tightly twisted return lead. Since there is no longer a low impedance pathway between the reference network and the signal circuit (the reference mesh is insulated from the body), loop L3 is broken, thus drastically reducing interference from that source.

Embodiments based on the equivalent circuit of FIG. 9 preferably utilise a mesh of carbon (or similar) wires or a preformed conductive fabric mesh located in the area of the signal electrodes (such as mounted on an electrode cap, insulated from the body) to provide a multitude of pathways for reference loops to match signal circuit loops. Further, these embodiments eliminate the third loop formed between the signal and reference wires, by virtue of isolating the reference circuits from the body, i.e., the reference leads are no longer connected to the earlobe. Further, the improved method provides a means of rejecting mains power interference by means of a separate signal circuit (with an isolated parallel reference loop) connected to the earlobe, subsequently subtracted from the EEG signal channels as described below.

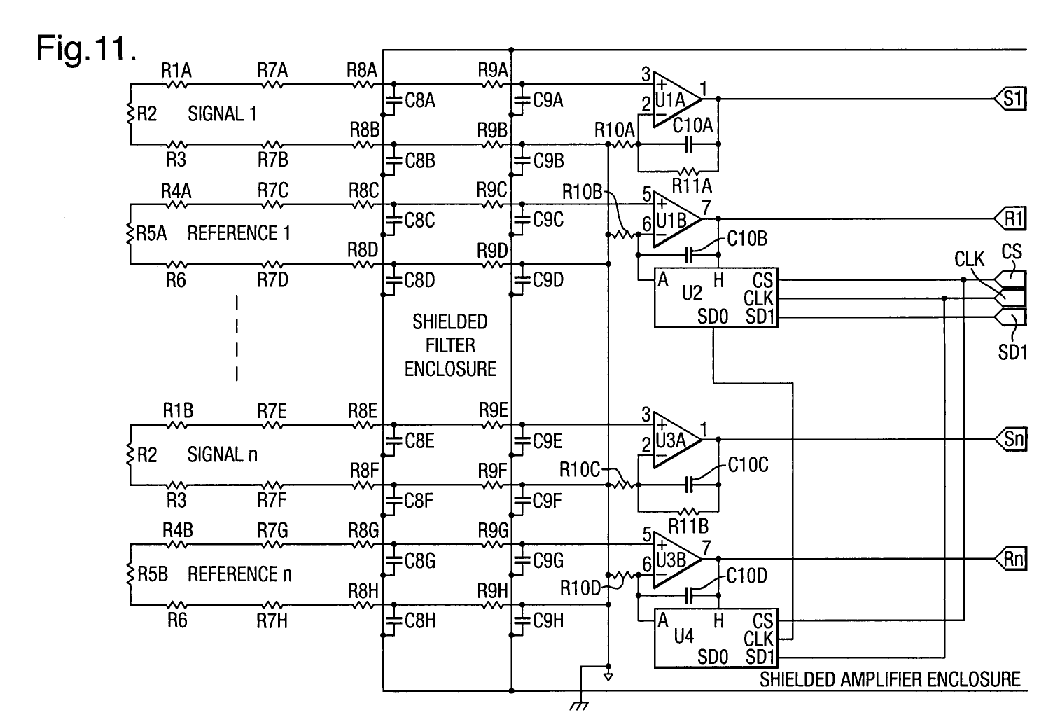

FIG. 10 shows part of an actual circuit comprising amplifiers and filters associated with a single channel of EEG for implementing the principles embodied in the equivalent circuits of FIGS. 7-9. The signal wire is connected to amplifier U1, and the reference loop associated with the signal line is connected to U2. Amplifier U1 is a high impedance low noise operational amplifier with fixed gain of 1 to 2. Amplifier U2 is also a high impedance low noise operational amplifier with variable gain controlled by a digital potentiometer allowing dynamic setting of the gain of U2 by software control for the purpose of matching the amplitude of induced interference voltage in the reference loop circuit with the induced interference voltage in the signal circuit. Alternatively, the gain of U2 may be matched to that of U1 by closely matching (to within 5% or less) the gain-setting components of the amplifiers. F1 and F2 are matched 2-pole low pass active filters with low overshoot characteristics such as a Bessel filter. Components R1, R2 and C1 form a low pass filter in combination with differential amplifier U3, which preferably maintains high common mode rejection at high frequency (for example, the AD8129 differential amplifier manufactured by Analog Devices, Inc., with a common mode rejection of 90 dB at 1 MHz).

The output of U3 is the desired signal with a gain of 10, minus the matched interference of the reference loop. Any mismatched interference in the signal and ref loops below the cutoff frequency of the low pass filters will be present. Mains powerline interference is also present at the output of U3. A means of reducing powerline interference in the signal is implemented by connecting a signal channel with accompanying reference loop to an earlobe or scalp site close to an ear. The signal from the earlobe (consisting primarily of induced powerline interference voltages from the human body) is connected to amplifier U4, and the associated reference loop is connected to U5.

F3 and F4 are matched filters similar to F1 and F2, and R3, R4 and C2 in combination with U6 (same type of differential amplifier as U3) form a low pass filter. U6 has a variable gain function implemented by means of a digital potentiometer under software control. The output of U6 is the powerline interference voltage minus the matched magnetic interference from the reference loop.

The outputs of U3 and U6 are fed into differential amplifier U7 which subtracts the mains interference from the EEG signal. Any mismatch between the mains interference voltages at the outputs of U3 and U6 may be minimized dynamically by adjusting the gain of U6 by software means, thus maximizing the cancellation of mains Hz interference in the EEG signal.

Amplifier U7 typically has a gain of 50, and the output is the amplified EEG signal with significant amounts of interference from magnetic (fMRI) and electrostatic (AC power) sources removed. Further amplification and filtering of the EEG may be implemented on the output of U7.

FIG. 10 thus shows a single channel implementation of the improved reference loop; in a multi-channel implementation the output of U6 is fed to the minus-inputs of the equivalent U7 amplifiers for all the EEG signal channels.

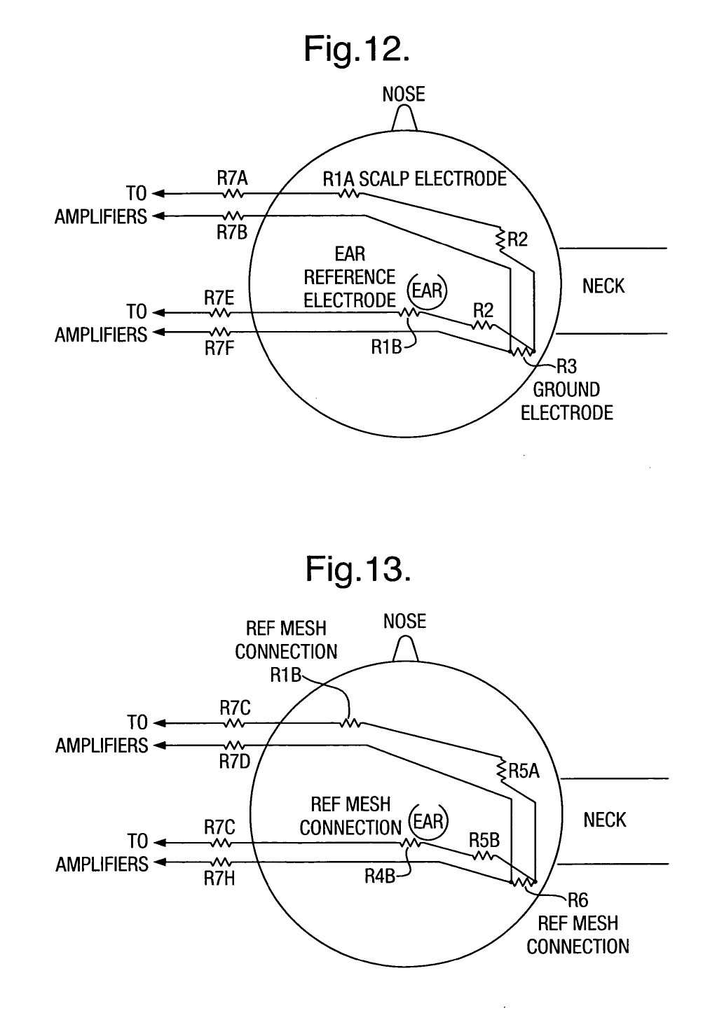

Another embodiment exemplifying apparatus and a method according to the present invention is shown in FIGS. 11-17.

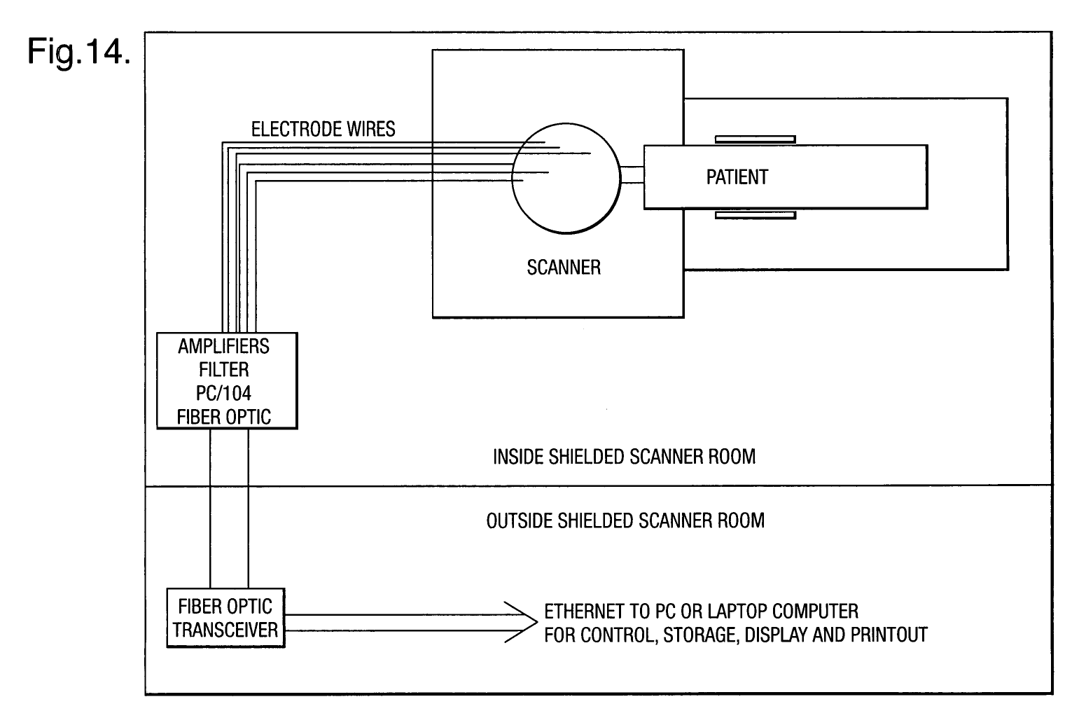

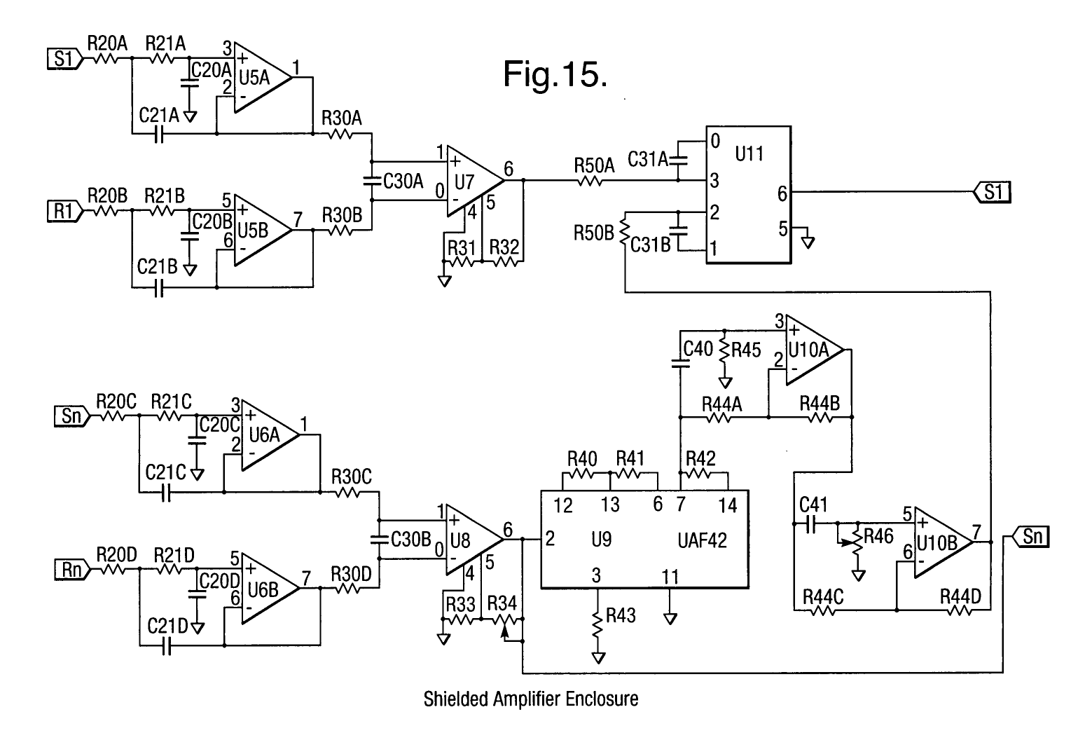

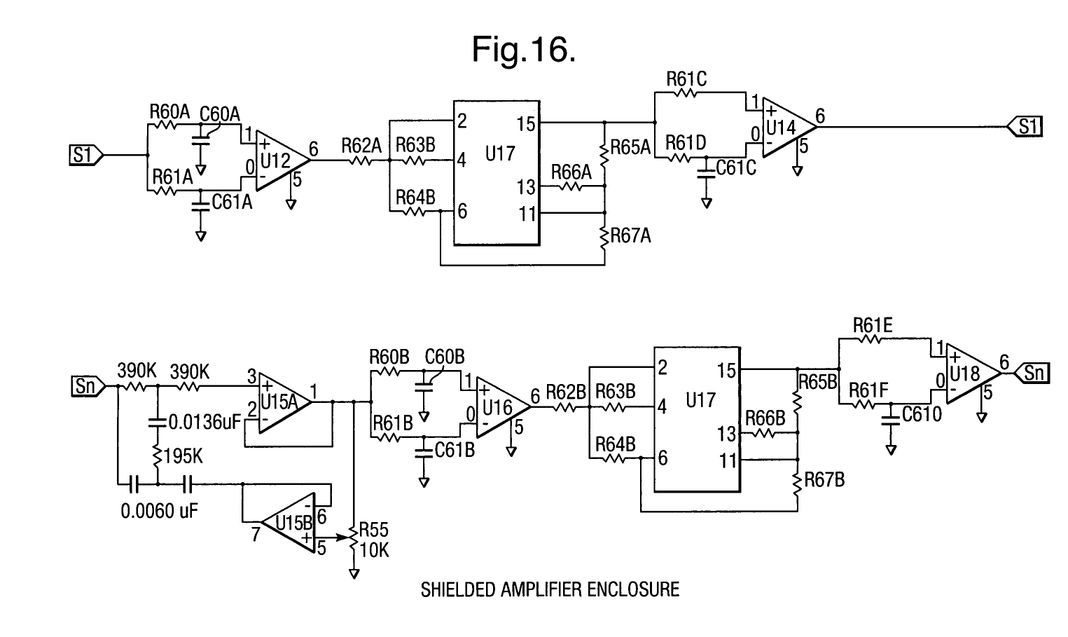

FIG. 11 shows the front end circuitry of this embodiment, which circuitry is attached to signal, reference and ground electrodes, which are attached to the subject who is inside the scan head within the scan room. FIGS. 12 and 13 show the electrode connections to the subject's head and the connections of the reference mesh, respectively. FIG. 14 shows the location of subject and system components with respect to the scan room. FIGS. 15, 16 and 17 show other circuitry details of this embodiment.

Referring to FIG. 11, there are n measurement channels, where n ranges typically from 2 to 1024. For convenience, only the 1^{st} and n'th channels are actually shown in the drawing. Each measurement channel comprises a signal line and a reference line. The signal line and reference line of each channel are paired with a respective ground line.

Thus, as shown, measurement channel 1 comprises signal line pair designated “Signal 1” and reference line pair “Reference 1”. As depicted, the signal line of “Signal 1”, is connected to the scalp for EEG via an electrode with an impedance represented by resistor R1A, preferably 10 K ohms or less. All body electrodes preferably are constructed of a resistive material such as carbon-loaded plastic, or the bare ends of carbon wire. Contact to the body is made via a conductive paste.

R2 represents the impedance of body tissue, typically 100 ohms. R3 represents the ground electrode, preferably 10K ohms or less, located typically at the base of the neck. Resistors R7 (A through H) represent the resistance of the carbon wire connecting the electrode or reference loop to the electronic amplifiers, combined with the resistance of a patient safety resistor. A typical value for R7 is 13K ohms. The safety resistor typically is 12.5K ohms (range 10 K to 15K ohms), preferably non-magnetic (such as Ohmite Macrochip™ SMD resistor), and is mounted in the electrode wire close (within 0.3 m) to the patient.

For channel 1 (and similarly for all signal channels), the wires represented by R7A and R7B are twisted together tightly to minimize the loop area formed by the wires and hence minimize induced magnetic field interference in the signal.

In measurement channel 1, R4A represents the connection of a carbon wire to a conductive reference mesh that spans the surface of the head but is not in electrical contact with the body. R4A is located very close to R1A. R5A represents the impedance of the reference mesh. R6 is the connection from the mesh to the return wire for the reference loop, represented by R7D. R6 is located very close to R3. The wires for the reference loop (R7C and R7D) are tightly together tightly to minimize loop area, and the pair is twisted together with the R7A-R7B pair to match the paths followed by the loops.

Preferably the impedances of R1A and R4A are matched, as well as those of R2 with R5A, and R3 with R6. However, it is acceptable if only the sums of impedances R1A+R2+R3 and R4A+R5A+R6 are reasonably matched. The signal appearing on the reference circuit is subtracted from the signal circuit. If impedances and wire pathways are well matched between signal and reference loops, the magnetically induced interference appearing in the signal circuit will be removed by subtraction of the reference signal.

All of the components associated with the reference mesh and body electrodes may be considered impedances (i.e. having to greater or lesser degrees, resistive, inductive and capacitive components). Thus, except where indicated explicitly to the contrary or where the context does not permit, as used herein, all references to resisitance may be regarded as including reference to impedance and “resistive” should be interpreted likewise.

The body electrodes (R1A-etc and R6) have significant resistive and capacitive elements down to about 10 Hz. R2, the body tissue beneath the scalp, may be considered to be solely resistive below 100 Hz. R4A-etc in the reference mesh corresponds to R1A-etc, and R5A-etc in the reference mesh corresponds to R2, with the goal being to match these corresponding elements electrically, primarily in the frequency range for physiological signals of interest, 1-1000 Hz. Above that range the electronic filters take over for eliminating magnetic and rf noise. There are capacitive and inductive elements in the reference mesh that are significant at rf, and matching the impedances of the loops at rf is desirable. However, for matching purposes, the maximum tolerable range may be considered to be a DC resistance measured in a reference mesh loop of 50 to 50K ohms. (Measured at the point where the loop connects to the cable, i.e., in front of resistance R7). A preferred range would be an impedance of between 1K and 10K ohms measured in the reference loop at a frequency of 10 Hz. The body electrode impedances (at 10 Hz) are preferably lower than 10K ohms with a maximum of 20K ohms measured between the signal electrode and ground electrode. Generally speaking, there is some level of electrical inter-connection between the points of connection to the reference mesh, depending on the construction. If a continuous conductive fabric or foam is used, there is significant connection throughout the material, and R5A-etc are all connected by primarily resistive and capacitive elements. At the other end of the spectrum, if a lattice network is used, then conductive strings connect the various junctions where R4A-etc. meet R5A-etc. Thus, “reference electrode” is to be interpreted as encompassing the extremes and all possible intermediate forms of construction. The connections are again primarily resistive and capacitive, and can be every junction connected to every other junction at one extreme, or at the other extreme just nearest neighbouring junctions connected.

The nth channel is connected to a neutral location (close to areas of physiological signals of interest but without signal activity) such as behind the ear or on the earlobe for EEG, and has the same configuration (as the signal channels) of a signal loop paired with a matching reference loop. R3 serves as a common ground electrode to the body for all signal circuits, and similarly R6 is a common ground connection to the reference mesh for all the reference circuits.

Thus, the overall electrical connection arrangement can be seen more clearly from FIGS. 12 and 13 with signal and reference (with respective ground) electrodes and connections disposed over the scalp of the subject (channels 1-(n-1)). In FIG. 12, the circuits track through the body whereas on FIG. 13, they track through a reference mesh in parallel with, but not through the body. The nth channel can be seen to comprise the last signal and reference electrodes and connections (with ground electrodes and connections) which are located beneath or on an ear. To repeat, the signal and ground electrodes are in low resistance contact with the skin whilst the reference electrodes (or connections) are part of the mesh which is close to but not direct (i.e. not in low resistance) contact with the skin.

The patient cable consisting of all carbon wires twisted in pairs is approximately 2 to 5 meters in length and terminates at the shielded enclosure containing rf filters, analog amplifiers, filters, A/D converters and digital control circuitry. Filtering for rf interference is accomplished with two layers of filters separated by a five-sided shielded enclosure (labelled “Shielded Filter Enclosure” in FIG. 11). The first rf filter begins with resistors R8, 100 to 1K ohms, carbon or thick film composition. Capacitors C8 represent feedthrough capacitors of 1000 pF to 10,000 pF inserted into the wall of the shielded filter enclosure. Attentively, capacitors C8 may be replaced by a filter connector such as Amphenol™ part number 21-474021-025 which has a pi filter configuration.

Resistors R9 begin the second rf filter (same values and types as R8), with feedthrough capacitors C9 (same values and types as C8) inserted into the wall of the shielded amplifier enclosure. Further rf filtering may be accomplished with the use of a 4-channel common mode choke for the four leads of each channel, and or the addition of a 100 to 1K ohm resistor followed by a 1 to 5 nF capacitor to ground in the leads to the non-inverting inputs of each preamplifier (pins 3 and 5 of U1 and U3 in FIG. 11), and or the insertion of a 100 to 500 pF capacitor between the inverting and non-inverting inputs of the preamplifiers.

Circuit power ground, or common, denoted by the triangle symbol within the shielded amplifier enclosure near the bottom of FIG. 11, is preferably connected to the metallic shield enclosure in one location as shown in the Figure but the shield may also remain isolated from circuit ground. Although circuit power connections are not shown in the Figures, it is understood that the analog integrated circuit amplifiers and filter IC's, etc., are connected to bipolar power supplies of typically ±2.5 volts to ±10 volts, and digital modules are connected to ±5 volts. Power is supplied preferably from batteries located within the shielded amplifier enclosure, but may also be supplied from an external power source (isolated medical grade power supply or batteries) if the power inputs are filtered for rf at the shield enclosure, using filters similar to those shown for the signal lines.

The preamplifiers (U1 and U3 in FIG. 11) are typically low noise, high input impedance dual operational amplifiers such as Analog Devices AD8620 or OP2177. On the signal side (U1A and U3A in FIG. 11) a gain of 2 (typical, range 1 to 4) is established by resistors R10 and R11, typically 33K ohms. On the reference side, variable gain is implemented by the use of a digitally controlled potentiometer (U2 and U4 in FIG. 11) in place of R11. This allows the dynamic adjustment of the reference signal gain under programmatic control for maximum interference reduction. Alternatively, R11 on the reference side may be a resistor matched to R11 on the signal side. High resolution is necessary for precision matching of signal levels in the channels; Analog Devices™ AD7376 with 128 positions, or Analog Devices AD5231 with 1024 steps are examples of digital potentiometers that may be used for U2 and U4. In one example, an AD7376 of 100K ohms is used with R10 and R11 equal to 33K ohms. In this instance, the signal gain is 2 and the reference gain varies from 1 to approximately 4. In another example, an AD5231 of 50K ohms is used with R10 and R11 equal to 17K ohms. In this case the signal gain is again two, and the reference gain varies from 1 to approximately 4, but the resolution of adjustment is greatly improved with 1024 steps instead of 128. In both cases, the control of the potentiometer is implemented via three digital control lines, labeled CS, CLK and SDI in FIG. 11. This method of control is desirable as it enables “daisy chaining” the digital potentiometers as shown in FIG. 11, which is advantageous for adjusting reference levels when large numbers of channels are used. Capacitors C10 reduce noise from the digital potentiometers when adjusting; they are used on the signal amplifiers to keep the bandwidths of the signal and reference amplifiers closely matched.

In FIG. 15, the signal and reference signals are filtered by second order Bessel filters constructed around U5 and U6, which are dual operational amplifiers of the same types as U1 and U3. The Bessel filters are low pass, with a cutoff (−3 dB) typically of 145 Hz. Resistors R20 and R21 are 6650 ohms, capacitors C20 are 0.12 uF and capacitors C21 are 0.22 uF for 145 Hz cutoff. The filters must be closely matched in each signal-reference pair to maintain high noise rejection at the differential amplifier; this is achieved by closely matching the filter components preferably to within 0.1% tolerance, or to a maximum of 1% tolerance.

Following the Bessel filters, a differential mode to common mode filter composed of resistors R30 and capacitors C30 (600 ohms and 1.0 μF respectively for a cutoff frequency of 133 Hz) is placed at the input of a wide bandwidth differential amplifier (U7 and U8 in FIG. 15) such as Analog Devices™ AD8129 or similar. The reference loop signal is subtracted at this stage, with an equivalent third order low pass filter of 100 Hz cutoff formed by the combination of filters and differential amplifier. Although low pass filtering is advantageous for minimizing interference, the signal and reference loops must be well-matched in order to minimize interference within the signal bandwidth, 100 Hz in this case.

The gain for the differential amplifier is typically set at 12.5. In FIG. 15, resistors R31 and R32 (221 ohms and 2.55K ohms respectively) set the gain for the signal channels. Channel n, connected to a neutral location on the body near the physiological signals of interest (such as the earlobe or behind the ear for EEG) is used for powerline interference reduction. After rf and magnetically induced interference is filtered and subtracted from channel n, the remaining signal (composed primarily of 50/60 Hz voltages capacitively coupled to the body from the power mains) is subtracted from the EEG signal. Therefore, channel n must be closely matched at 50/60 Hz to the EEG channels, and an adjustable gain control at differential amplifier U8 in FIG. 15 enables matching the gain of channel n to the other channels. The gain range for U8 is set by R33 at 221 ohms, and R34, a 2490 ohms resistor in series with a 100 ohms potentiometer. For maximum powerline rejection, a variable gain control may be added to each EEG channel for individual adjustment, such as replacing R32 with a 2490 ohms resistor in series with a 100 ohms potentiometer.

Since the signal on channel n is subtracted from the other signal channels, any residual interference appearing on channel n from sources other than 50/60 Hz powerline voltages will appear on the signal channels if it is not matched to the interference on each signal channel. Precise matching of residual interference across channels is not expected, so a means of minimizing any signal other than powerline noise appearing on channel n is necessary.

One method, shown in FIG. 15, is to bandpass filter channel n with a Texas Instruments™ UAF42 filter IC (U9) set at 50 or 60 Hz. For a center frequency of 60 Hz, Q equal to 30, and bandpass gain of 1, R40 is set to 5.49K ohms, R41 and 42 are 834K ohms, and R43 is 487 ohms. Phase adjustment is necessary after filtering to precisely match the phase of the 50/60 Hz signal remaining on channel n to the other signal channels. In FIG. 15, this is implemented with two all pass filter circuits constructed around dual operational amplifier U10 (Texas Instruments TL072 or similar). For 90 degrees of phase shift at 60 Hz, capacitors C40 and C41 are set to 1 μF. Resistor R45 is 265K ohms and resistor R46 is a combination of 261K ohms in series with a 10K ohms potentiometer for phase adjustment. Resistors R44 are 100K ohms. Alternatively, R46 may be replaced with a digitally controlled potentiometer as described above for adjusting amplifier gains, in order to adjust phase shift by programmed means.

An alternative approach is to use a bandpass filter with lower Q to allow a passband of 50 to 60 Hz, and follow with a phase locked loop to lock onto the powerline noise. The output of the phase locked loop is phase adjusted and the gain may be trimmed to match the powerline interference appearing on the signal channels. The filtered and phase adjusted powerline interference signal on channel n is subtracted from the signal channels using a differential amplifier (U11 in FIG. 15, Analog Devices AD620 or similar). Resistors R50 (1K ohms) and capacitors C31 (150 pF) filter high frequency noise appearing at the output of the wide bandwidth differential amplifier U7, and match the inputs at U7.

In FIG. 16, the main stages of signal amplification and additional filtering are shown. At the input to U12 (differential amplifier such as Analog Devices™ AD627), the signal channel is high pass filtered to remove DC offsets appearing at the electrode interface to the body. Typical values for components are: R60, 39.2K ohms, R61, 1.6M ohms, C60, 0.01 uF, and C61 0.1 uF. Gain for this stage is set at 10. Following is a fourth order Butterworth low pass filter with a cutoff frequency of 256 Hz. This may be implemented using a Linear Devices™ LTC1563-2 filter (U13 in FIG. 16) with resistors R62 through R67 set to 10M ohms. Additional gain of 50 and DC offset filtering is added at U14 and U18 (AD627 typically) with R61 set to 1.6M ohms and C61 at 0.1 uF.

Although all channels have the same amplification and filtering as outlined above, channel n has an additional filter as shown in FIG. 16. Since channel n is the powerline reference channel, the primary signal appearing on this channel is a large 50/60 Hz signal. As previously described, this signal is subtracted from the signal channels to remove powerline interference. However, in some applications, it may be necessary to observe channel n in order to adjust the reference loop gain for minimizing rf and magnetically induced interference. Therefore, the original channel n signal appearing at the output of U8 in FIG. 15 is routed through a 50 or 60 Hz notch filter in FIG. 16 before amplification and digitization for display. A 60 Hz notch filter is built around operational amplifier U15 (Texas lnstruments™ TL072 or similar) using component values shown in FIG. 16, resulting in approximately 45 dB of rejection at 60 Hz, sufficient for displaying channel n without excess powerline noise swamping the trace.

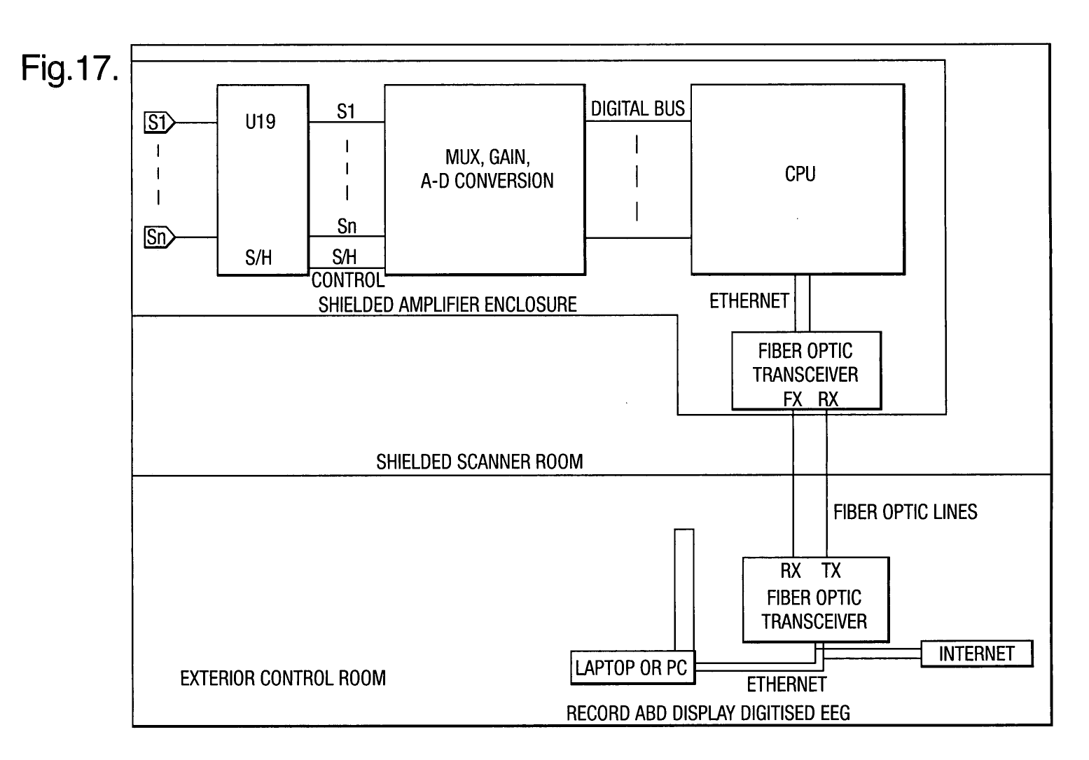

In FIG. 17, the final components of the system are shown. U19 represents sample and hold amplifiers for each channel, enabling simultaneous sampling for all channels to avoid distortion of signal samples due to time skewing. After further optional gain adjustment, the sampled signals are digitized to 16 bit resolution. A commercially available 32 channel analog I/O module such as Diamond Systems™ Diamond-MM-32-AT on a PC/104 bus may be used for analog to digital conversion. Further digital control is performed using a CPU such as a Diamond Systems Promethius™ PC/104 CPU module. Software for controlling timing of sampling, digitization, communication over ethernet and other functions is loaded into the PC/104 CPU module.

Communication with the external world is accomplished via Ethernet, with a fiber optic link inserted to avoid conducting interference into the shielded MRI scanner room on metallic wires. The fiber optic link also minimizes rf interference leaking into or out of the shielded amplifier enclosure, and for patient safety isolates the amplifier electronics from AC power leakage through the network connection. Fiber optic conversion may be accomplished using a Telebye™ Model 373 10Base-T (Ethernet) to Fiber Optic Transceiver. Communication with the PC/104 CPU via networking enables command of the system from remote locations (such as the MRI control room or other office) and allows data to be delivered to multiple locations for recording, display and analysis (anywhere on the internet, essentially). Commands from external computer control initiate functions of the PC/104 CPU, including sampling, reference gain, powerline gain and phase adjust, real time data display, data dump for permanent recording, etc. Although data is temporarily stored in the PC/104 CPU, it must be transferred to data storage such as a computer hard drive for permanent recording.

In the light of the described embodiments, modifications of those embodiments, as well as other embodiments, all within the scope of the appended claims as interpreted in the light of the specification as a whole and with the knowledge of a person skilled in the art, will now become apparent.

1. An electronic apparatus for reducing interference in a desired signal, the apparatus comprising
(a) a signal line connected to a signal electrode; and
(b) a reference line connected to a reference electrode;
said signal line and reference line being associated by being in close physical proximity for a substantial part of their lengths, said electronic apparatus further comprising subtraction means for subtracting an interference signal on the reference line from an interference signal on the signal line thereby to enhance a desired signal on the-signal line.

2. The electronic apparatus of claim 1, wherein the signal electrode is in direct electrical contact with a subject and the reference electrode is in close proximity to but not in direct electrical contact with the subject.

3. The electronic apparatus of claim 1, wherein a ground line is arranged in associated close proximity with the signal line along a substantial part of the length thereof, and a further ground line is arranged in associated close proximity with the reference line along a substantial part of the length thereof, each of the ground lines being connected to one or more ground electrodes in direct electrical contact with the subject.

4. The electronic apparatus of claim 1, comprising one or more additional signal lines, each connected to a respective additional signal electrode, each additional signal line having a respective associated additional reference line in close physical proximity therewith, for a substantial part of its length so that the signal line and each additional signal line with their corresponding reference lines form respective signal line/reference line pairs, the reference line and each additional reference line being connected to said reference electrode or respectively to said reference electrode and to one or more of additional reference electrodes, wherein said subtraction means is arranged to subtract a respective interference signal on the reference line and each additional reference line from a respective interference signal on the signal line with which that reference line is associated, thereby to enhance a desired signal on the signal line and a desired signal on each additional signal line.

5. The electronic apparatus of claim 3, wherein each additional signal line and each additional reference line has a respective additional ground line arranged in close proximity therewith along a substantial part of their respective lengths, each of the additional ground lines being connected to the ground electrode or electrodes or to one or more additional ground electrodes in direct electrical contact with the subject.

6. The electronic apparatus of claim 5, wherein each signal line and associated ground line are respectively twisted together and each reference line and associated ground line are respectively twisted together.

7. The electronic apparatus of claim 5, wherein each signal line/ground line twisted pair and each associated reference line/ground line twisted pair are respectively twisted together.

8. The electronic apparatus of claim 1, comprising one or a plurality of signal line/reference line pairs; wherein each signal line/reference line pair is shielded.

9. The electronic apparatus of claim 8, wherein said subtraction means comprises a common mode choke for the or each signal line/reference line pair such that the signal line and reference line of each pair are connected to respective windings of their respective common mode choke.

10. The electronic apparatus of claim 1, wherein the subtraction means comprises low pass filter means.

11. The electronic apparatus of claim 10, wherein the low pass filter means comprise a second order low pass filter.

12. The electronic apparatus of claim 10, wherein the low pass filter means is of Bessel type.

13. A combined fMRI and EPM apparatus comprising an MRI unit and an EPM system which comprises an electronic apparatus for reducing interference in a desired signal, the apparatus comprising
(a) a signal line connected to a signal electrode; and
(b) a reference line connected to a reference electrode;
said signal line and reference line being associated by being in close physical proximity for a substantial part of their lengths, said electronic apparatus further comprising subtraction means for subtracting an interference signal on the reference line from an interference signal on the signal line thereby to enhance a desired signal on the signal line.

14. The combined apparatus of claim 13, wherein the EPM system is selected from systems for effecting one or more of EEG, ECG, EMG, EOG, ERG and GSR.

15. A method of reducing interference from a designed signal, the method comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the interference signal on the reference line from the interference signal on the signal line.

16. The method of claim 15, wherein the signal electrode is in direct electrical contact with a subject and the reference electrode is in close proximity to but not in direct electrical contact with the subject.

17. The method of claim 16, wherein a ground line is arranged in associated close proximity with the signal line along a substantial part of the length thereof, and a further ground line is arranged in associated close proximity with the reference line along a substantial part of the length thereof, each of the ground lines being connected to one or more ground electrodes in direct electrical contact with the subject.

18. The method of claim 15, wherein one or more additional signal lines are provided, each additional signal having a respective additional reference line in close physical proximity therewith, for a substantial part of its length, the reference line and each additional reference line being connected to said reference electrode or respectively to said reference electrode and to one or more additional reference electrodes, and wherein the subtraction step further comprises subtraction of a respective interference signal on each additional reference line from a respective desired signal on its associated signal line.

19. The method of claim 17, wherein each additional signal line and each additional reference line has a respective additional ground line arranged in close proximity therewith along a substantial part of their respective lengths, each of the additional ground lines being connected to the ground electrode or electrodes or to one or more additional ground electrodes in direct electrical contact with the subject.

20. The method of claim 19, wherein each signal line and associated ground line are respectively twisted together and each reference line and associated ground line are respectively twisted together.

21. The method of claim 20, wherein each signal line/ground line twisted pair and each associated reference line/ground line twisted pair are respectively twisted together.

22. The method of claim 15, wherein each signal line is connected to a respective signal electrode and each reference line is connected to one or more reference electrodes.

23. A method of performing a simultaneous fMRI and EPM, the method comprising placing a subject in an MRI apparatus and performing MRI scans and simultaneously taking EPM measurements by a method which comprises reducing interference from a designed signal by steps comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the interference signal on the reference line from the interference signal on the signal line.

24. An electronic apparatus for reducing interference in a signal derived from an EPM, the apparatus comprising
(a) a signal line connected to a signal electrode;
(b) a reference line connected to a reference electrode; and
(c) at least one ground line for said signal line and reference line, said ground line or lines being connected to a ground electrode or individually to respective ground electrodes;
said electronic apparatus further comprising subtraction means for subtracting an interference signal on the reference line from a signal on the signal line.

25. The apparatus of claim 24, comprising one or more additional signal lines, each connected to a respective additional signal electrode, each additional signal line having a respective associated additional reference line, a respective additional ground line connected to said ground electrode or electrodes or to a respective additional ground electrode being provided for each additional signal line and each additional reference line, so that the signal line and each additional signal line with their corresponding reference lines form respective signal line/reference line pairs, the reference line and each additional reference line being connected respectively to said reference electrode and to one or more of additional reference electrodes, wherein said subtraction means is arranged to subtract a respective interference signal on the reference line and each additional reference line from a respective interference signal on the signal line with which that reference line is associated, thereby to enhance a desired signal on the signal line and a desired signal on each additional signal line.

26. The apparatus of claim 24, wherein each signal line and a respective associated reference line are in close physical proximity for a substantial part of their lengths.

27. The apparatus of claim 24, wherein the signal line and its associated ground line and each reference line and its associated ground line are associated by being in close physical proximity for a substantial part of their lengths.

28. A method of reducing interference on a signal derived from an EPM, the method comprising
(a) providing a signal line carrying a desired signal and a first interference signal, said signal line being connected to a signal electrode;
(b) providing a reference line carrying at least a second interference signal, said reference line being connected to a reference electrode;
(c) providing at least one ground line for said signal line and reference line, said ground line or lines being connected to at least one ground electrode or individually to respective ground electrodes; and
(d) a subtraction step of subtracting the second interference signal on the reference line from the first interference signal on the signal line.

29. The method of claim 28, comprising one or more additional signal lines, each connected to a respective additional signal electrode, each additional signal line having a respective associated additional reference line, a respective additional ground line connected to said ground electrode or electrodes or to a respective additional ground electrode being provided for each additional signal line and each additional reference line, so that the signal line and each additional signal line with their corresponding reference lines form respective signal line/reference line pairs, the reference line and each additional reference line being connected respectively to said reference electrode and to one or more of additional reference electrodes, wherein said subtraction means is arranged to subtract a respective interference signal on the reference line and each additional reference line from a respective interference signal on the signal line with which that reference line is associated, thereby to enhance a desired signal on the signal line and a desired signal on each additional signal line.

30. The method of claim 28, wherein each signal line and a respective associated reference line are in close physical proximity for a substantial part of their lengths.

31. The method of claim 28, wherein each signal line and its associated ground line and each reference line and its associated ground line are associated by being in close physical proximity for a substantial part of their lengths.

32. An electrode support structure apparatus for effecting an EPM, the apparatus comprising an electrode support having supported thereon, an array of signal electrodes presented for contacting the skin of a subject, first connection means being provided for independent electrical connection to each of said signal electrodes, the apparatus further comprising an electrically insulated mesh having one or more of reference nodes and second connection means for independent electrical connection to the or each of said reference nodes.

33. The apparatus of claim 31, wherein the number of said reference nodes is substantially the same as the number of said signal electrodes.

34. The apparatus of claim 32, wherein each signal electrode has a corresponding respective reference node in close physical proximity thereto.

35. The apparatus of claim 32, wherein said electrode support further supports one or more ground electrodes presented for contacting the skin of a subject, the apparatus further comprising third connection means for independent electrical connection to each of said ground electrode or electrodes.

36. The apparatus of claim 35, wherein the number of said ground electrodes is substantially equal to the number of said signal electrodes plus the number of said reference nodes.

37. The apparatus of claim 35, wherein each signal electrode and each reference node has a corresponding respective ground electrode in close physical proximity thereto.

38. The apparatus of claim 35, wherein the number of reference nodes is substantially equal to the number of signal electrodes and is also substantially equal to the number of ground electrodes.

39. The apparatus of claim 32, wherein said reference mesh comprises a continuous laminar member comprising said reference nodes.

40. The apparatus of claim 32, wherein said reference mesh comprises a matrix of discrete members respectively comprising said reference nodes.

41. The apparatus of claim 32, wherein said electrode support is in the form of a flexible cap.
